(19) United States
(12) Patent Application Publication
(10) Pub. No.: US 2026/0345190 A1
(43) Pub. Date: Nov. 5, 2026

(54) CUTTING DEVICE AND METHOD FOR COMMINUTING BIOLOGICAL MATERIAL IN A HOLLOW ORGAN

(71) Applicant: MedTecton GmbH, Horgen (CH)

(72) Inventors: Kurt RUFFIEUX, Horgen (CH); Bernhard HUG, Freiburg (DE); Arkadiusz MIERNIK, Freiburg (DE)

(21) Appl. No.: 18/997,529

(22) PCT Filed: Jul. 5, 2023
(86) PCT No.: PCT/EP2023/068604

§ 371 Date: Jan. 22, 2025

(30) Foreign Application Priority Data
Jul. 22, 2022 (DE) 10 2022 118 416.1

(51) Int. Cl. A61B 17/32; A61B 17/00

(52) U.S. Cl. CPC A61B 17/32002; A61B 2017/00026; A61B 2017/320024; A61B 2217/005

(57) ABSTRACT

The invention relates to providing, on a cutting device (1) comprising a mechanically drivable cutting head (8), formed on a distal end (6) of a shaft (7), and a control unit (10), a proximity sensor (13) at the distal end (6) of the shaft (7). According to the invention, this cutting device (1) is to be used as part of a method for comminuting biological material (2) inside a hollow organ (3) (see FIG. 1). This makes it possible to activate or switch off the cutting head on the basis of an approach, for example even contact, detected by the proximity sensor.

### CROSS-REFERENCE TO RELATED APPLICATIONS

Reference is made to PCT/EP2023/068604 filed Jul. 5, 2023, and German Application No. 10 2022 118 416.1, filed Jul. 22, 2022, which are incorporated herein by reference in their entirety.

### BACKGROUND OF THE INVENTION

### Field of the Invention

The invention relates to a cutting device with a mechanically drivable cutting head formed on a distal end of a shaft and a control unit.

The invention also relates to a method for comminuting biological material within a hollow organ.

### Description of the Prior Art

Such cutting devices are used, for example, in the form of a morcellator in order to be able to remove resected material, that is tissue fragments, from hollow spaces.

Such resected materials can occur in the case of urological operations, for example a prostate operation or prostate intervention, when organ parts are removed as part of this operation or intervention. For example, in such a surgical intervention, the shaft of a cutting device, for instance a morcellator, can be introduced into the patient via the urethra and then advanced into the area of the prostate and the bladder. Tissue fragments can be peeled from the prostate and pushed into the bladder. These tissue fragments are generally too large to be removed from the patient via the urethra. A cutting device, for example a morcellator, can then be used to comminute and suck away the tissue fragments. The tissue fragments can thus be removed from outside without opening the bladder, which is particularly gentle on the patient. The cutting head can comprise a blade that rotates and/or oscillates, for example However, in connection with such cutting devices there is the danger that tissue can be cut unintentionally. For example, it can be the case that the wall of the hollow space, for example of the hollow organ, more particularly the bladder, in which the biological material is being comminuted, is accidentally contacted and cut with the cutting device. This can also occur if, for example, this tissue comes into the catchment range of a suction device.

### SUMMARY OF THE INVENTION

The aim of the invention is to avoid such incidents.

According to the invention, to solve this task in cutting devices, at the distal end of the shaft, a probe of a proximity sensor is formed by a metallic part of the shaft. A special case of the proximity sensor is thus the contact sensor which detects an approach to a distance of zero.

Since the probe is formed by a metallic part of the shaft, the distal end of the shaft itself can take over this sensor function. The probe is therefore not a separate component but is identical to at least one part of the shaft.

By use of the proximity sensor, it is therefore possible to detect an approach to material that is not to be cut. This can be the tissue of a hollow organ. This can therefore bring about the precondition for preventing unintentional cutting of the hollow organ. The proximity sensor can be an electrode, for example. The proximity sensor can also be designed in the form of a conductive section of the shaft.

Proximity sensors based on optical, pressure-based, capacitive or (ultra)sound-based operating methods can also be used. In each case the probe then comprises an appropriate sensor. However, the preferred embodiment is the impedance-based or electrical implementation that is described in detail.

The invention can in principle be used for cutting tissue in hollow spaces. The description of the invention and its advantages will above all deal with the embodiment of the invention for cutting biological tissue in hollow organs of a human patient as part of a surgical, more particularly urological, intervention. However, the invention is not restricted to this.

In an advantageous embodiment the metallic part of the shaft can form the distal end of the shaft.

The application of separate probes on or in the shaft can thus be eliminated. An outermost distal end of the shaft can therefore even be used for detection without a separate probe having to be applied thereto. In contrast to separate probes that are not applied to the distal end of the shaft but to another position of the shaft, it is thus advantageous that tissue detection can take place earlier, for example still before any tissue contact. This increases the safety of the cutting device according to the invention.

In an advantageous embodiment, the metallic part of the shaft provides mechanical stability of the shaft.

Therefore, separate stability-providing parts on or in the shaft are eliminated. In this way the cutting device according to the invention can be manufactured more compactly and with fewer components, which reduces the manufacturing and maintenance costs. Here, the mechanical stability can be understood in particular as a degree of stability provided by the metallic part, without which the shaft would be at least one of unstable and not useable without further stabilizing aids.

In an advantageous embodiment, the metallic part of the shaft accommodates a blade of the cutting head in a manner forming a housing.

Thus, the metallic part, forming the probe, can at the same time form a housing of a blade of the cutting head. In this way the blade can be protected from outside influences and the surroundings of the shaft from damage by the blade.

In an advantageous embodiment the control unit can be configured to control the cutting head in the case of an approach detected by the proximity sensor.

Alternatively or additionally, it can be that the control unit is set up to switch off the cutting head in the case of an approach, in particular contacting, detected by the proximity sensor.

In this way, controlling or switching off of the cutting head can take place on the basis of an approach, for example even contact, detected by the proximity sensor. Tissue damage can be avoided.

In an advantageous embodiment, a suction device is provided that is connected in a fluidic manner to a suction opening preferably formed on the distal end of the shaft.

At the distal end of the shaft, biological material comminuted by the cutting device, can thus be sucked in by the suction device and a tissue part can then cut off and this then sucked away.

The addition of a fluid, more particularly rinsing liquid, can take place through a rising channel of an endoscope in which the cutting device can be carried, preferably in an operating channel, in a longitudinally displaceable manner. The addition can be at least one of actuated and regulated by the suction process, for example.

In this way, for example rising in the area of the distal end of the shaft, for example rinsing of the hollow organ with a fluid, preferably with a liquid, can take place. Through the regulated addition of liquid, the formation of a negative pressure in the hollow organ that can lead to collapse of the hollow organ, is prevented.

The rinsing line or the rinsing channel can preferably extend through an endoscope, wherein the rinsing line or the rinsing channel extends through the interior of the shaft, for example.

A disadvantage of this suction is that when using the shaft in the vicinity of a wall of a hollow space or hollow organ, this wall, or the tissue that forms the wall, can be sucked in and thus be injured by the cutting device. The invention makes it possible to avert this situation by switching off the cutting device before substantial tissue damage is caused.

In an advantageous embodiment the control device is set up to control the suction device in the case of an approach detected by the proximity sensor, more particularly contact sensor.

Alternatively or additionally, it can be that the control unit is configured to switch off the suction device in the case of an approach detected by the proximity sensor.

Thus, controlling or switching off of the suction device can take place on the basis of an approach, even a contact, for example even a contact, detected by the proximity sensor.

In an advantageous embodiment, the control unit is configured to control the cutting head in reverse in the case of an approach detected by the proximity sensor.

For example, a blade of the cutting head can be moved backwards. A blade of the cutting head can also be moved in the direction of a neutral position. Simply switching off the cutting head can lead to contact, but not yet cutting tissue remaining caught in the cutting head and being damaged when the cutting head is pulled back from the tissue. The risk of such entrapment can be reduced or averted through controlling the cutting head in the opposite direction.

In an advantageous embodiment the control device is configured to control the suction device in reverse in the case of an approach detected by the proximity sensor.

For example, this control can result in the suction device emitting a fluid at its suction opening. In this way, trapped tissue or also remnants of the cut biological material can be removed from the area of the cutting head. The suction opening at the distal end of the shaft can be appropriately configured for this, for example.

All the aforementioned types of control can be implemented individually or in combination with each other, thus, for example, cutting and suction at the same time.

In an advantageous embodiment the control device is connected to a measuring electrode that can be applied to a part of the body.

Signals of the measuring electrode can be used to determine the approach of the proximity sensor, hence also of the cutting head. The measuring electrode can be applied to the patient being treated, for example. This can take place by a non-invasive procedure, for instance at least one of on the shaft of the endoscope and directly onto the patient's skin. In this way, to determine the approach, an electrical connection is established between the proximity sensor and the measuring electrode.

In an advantageous embodiment, one section the (morcellator) shaft is electrically insulated vis-a-vis the surroundings. This can take place, for example, in that electrical insulation is applied on the outer side of the shaft.

However, the electrical insulation can also be applied on a side of a tube, for example of an endoscope, encompassing the shaft. In this way, for example, an electrically insulated endoscope can be used without a further insulated (morcellator) shaft.

For instance, it can be that an operating channel of an endoscope accommodating the cutting device is insulated on the inside. Alternatively or additionally, it can be envisaged that an endoscope is insulated on the outside. In this way, a current flow to the cutting device at an access opening of a treatment area can be prevented.

It is advantageous if the insulation is made of a material having sliding properties facilitating the introduction of the cutting device into the patient or into the endoscope. The insulation of the shaft in one section can also be designed so that when correctly used, the section is positioned at an access opening of an intervention space. This is the space in which the surgical intervention takes place. It can also be a shaft made of an insulating material or a shaft, fitted with electrical wires in its interior, that is completely (possibly with the exception of a distal end area) covered with insulating material. Through these measures, an electrical contact between the cutting device and the patient's body outside the hollow organ can be avoided.

In an advantageous embodiment, a connection for diverting a current from the distal end of the shaft is formed at the proximal end of the shaft.

In this way, a signal at the distal end of the shaft can be picked up by the proximal end of the shaft and used, for example, for detecting the approach, more particularly contact, in particular of the cutting head on the tissue of the hollow organ. Thus, when the cutting device is being used as intended, a signal (e.g. sensor current) from the interior of the hollow organ can be picked up from outside the patient's body and processed further.

It can also be that the shaft is electrically connected to the control unit.

The signal picked up by the proximal end of the shaft can therefore be transmitted directly to and processed by the control unit.

In an advantageous embodiment, it can be envisaged that there is an electrical generator of a monitor current.

The monitor current can, for example, be applied to the distal end of the shaft. For this, alternating current with a frequency of between 30 KHz and 35 KHz is preferably used. Alternating current of such frequencies has a particularly small effect on the propagation of nerve signals in the body. The use of direct current is also possible.

In an advantageous embodiment it can be that an electrical resistance between the distal end of the shaft and a measuring electrode applied on a patient is determined. Alternatively, an endoscope shaft can also be used as a measuring electrode.

Here, the invention uses the fact that an impedance between the distal end of the shaft and a measuring electrode applied to the patient becomes smaller with increasing approach of the distal end to the tissue of the patient's hollow organ. For example, a threshold value can be defined, as of which controlling/switching off of the cutting head and/or the suction device should take place. In this way, the simple approach of the distal end to the tissue of the hollow organ can already be responded to, without a contact having to occur. Comminution of the tissue in the hollow organ can thus be made safer than if switching off were only to take place in the event of contact with the tissue. The danger of unintentional cutting of tissue can thereby be reduced or averted.

In an advantageous embodiment it can be that an impedance (complex electrical resistance) between the distal end of the shaft and a or the measuring electrode is determined using high-frequency alternating current.

In this way, the impedance can be determined using the high-frequency alternating current provided by the generator. Here, alternating current with a frequency of between 30 and 35 KHz can preferably be used. Determining the approach can take place without significantly influencing the propagation of nerve signals in the body and thus in a particularly gentle manner for the patient.

In an advantageous embodiment it can be that when used as intended, the distal end of the shaft is surrounded by at least one of an electrically weakly conductive and non-conductive fluid. The fluid is preferably a liquid. It can also be that that at least one of electrically weakly conductive and non-conductive fluid is in contact with a biological tissue.

In this way, the hollow organ is at least partially filled with the electrically weakly conductive and non-conductive liquid. This serves to generate as great as possible a difference in the impedance between the situation of the electrode placed freely in the hollow organ and the situation where the electrode contacts the tissue of the hollow organ. Available weakly conductive and non-conductive liquids that are available in everyday clinical protocol can be used. Available electrically weakly conductive and non-conductive liquids, pre-prepared liquids (electrolyte-free solutions) used on everyday claimed protocols are available, they can be used. A widely-used liquid of this type is the product Purisole, supplied by Fresenius-Kabi company. These liquids can at the same time be matched to further requirements as part of the surgical intervention. This can involve maintaining an advantageous osmotic pressure.

In an advantageous embodiment, a memory unit is provided and configured to store at least one reference value for an impedance between the distal end of the shaft and a or the measuring electrode, wherein the at least one reference value is used for detecting the approach.

The electrical resistance can be the impedance. The impedance can, for example, be that between the proximity sensor and a or the measuring electrode. Here, at least one of a proportion of the patient's body and the cutting device in the impedance can be compensated for by calibration with reference values, for example.

In this way, when the cutting device is being used as intended, a patients individual resistance value can be used as the reference value. This can be determined, for example, through contacting the wall of the hollow organ with the cutting head switched off. During the use of the cutting head, a resistance value approaching the reference value can then be interpreted as an approach.

In an advantageous use, it can be that with the cutting device at least two signal paths are created, which can be used for determining at least two reference values for an impedance between the distal end of the shaft and a or the measuring electrode. In this way, reference values can be recorded through the liquid or the fluid, and in the case of bridging the liquid or the fluid through contacting bodily tissue, for example for a calibration, more particularly the previously mentioned calibration. For example, a first reference value can be recorded in that the (preferably deactivated) cutting device is brought into contact with the patient's tissue. Through this, a signal path is formed in which the liquid is short-circuited. A second reference value can be recorded, for example, in that that cutting device is removed from the patient's tissue, for instance retracted into an interior of the liquid or the fluid. In this way a second signal path is formed, which leads through the liquid or the fluid. Influences of a (complex) conductivity of at least one of the patient and the cutting device can be reduced or even eliminated.

The impedance can be that between the proximity sensor and for example as already mentioned, the measuring electrode.

In this way, a further individual patient resistance value can be used as the reference and included in the detection of the approach. In general, two different current paths can be generated with the associated resistance values of which being used as reference values. For example, one reference value can be determined on contacting the wall of the hollow organ, and a further reference value without contacting the hollow organ and, for example, only in the case of a proximity sensor surrounded by an only at least one oft he electrically weakly conductive and non-conductive liquid. A resistance value moving from one reference value to the other reference value can then be interpreted as an approach.

In this way, the aforementioned advantages of the cutting device according to the invention can be utilized in the case of the methods. This can involve a surgical intervention, preferably on a human patient.

In an advantageous embodiment, the biological material is comminuted by the cutting head.

Alternatively or additionally, it can be that the biological material is sucked in at least one of the suction device and transported away from the hollow organ.

In this way, at least one of the cutting heads and the suction devices introduced into the body by the cutting device according to the invention can be used to comminute the removed tissue.

In an advantageous embodiment the measuring electrode is applied from outside on the upper leg or in the abdominal area of the patient.

In an advantageous embodiment it can be that the hollow organ is at least partially filled with at least one of an electrically weakly conductive and non-conductive fluid.

The reliability of detection of the approach can thus be increased. This applies in particular in the case of detection of the approach by an electronic method, such as, for example, the determination an electrical impedance. A direct current flow between the distal end of the shaft of the cutting device and the wall of the hollow organ that could trigger a false detection of an approach, can thus be reduced or prevented.

Here, it can additionally be that a filling level of the hollow organ with the fluid is determined by measuring an impedance between the distal end of the shaft and a or the measuring electrode.

The filling level can, for example, relate to an absolute filling capacity of the hollow organ. However, the filling level can also relate to an absolute or relative amount of at least one of the substance and the volumetric proportion of the electrically weakly conductive non-conductive fluid present in the hollow organ.

For example, while implementing a method according to the invention, it may become necessary to replace an electrically conductive fluid with a less electrically conductive fluid in the hollow organ. The electrically conductive fluid can, for example, also be introduced into the hollow organ during a previous surgical procedure. At least one of the electrically weakly conductive and non-conductive fluid can, for example, be filled into the hollow organ via a rinsing line or rinsing channel as previously described. At the same time, the fluid present in the hollow organ can, for example, be removed from the hollow organ by a suction device as previously described. The progress of this fluid exchange, or filling of the hollow organ with the fluid, can be monitored by determining an impedance, for example, as previously described. Topping up of at least one of the electrically weakly conductive and non-conductive fluid can take place until the conductivity of the fluid present in the hollow organ, for example a mixture of fluids, has fallen below a certain measure. This state can be detected with the described impedance measurement.

In an advantageous embodiment it can be that at least two reference values for an impedance between the distal end of the shaft and the measuring electrode are determined in that at least one of two different signal paths are used and generated.

In doing so, it can, for example, be that the first signal path is formed by a fluid, for example a rinsing fluid, surrounding the probe. In this way a reference value can be generated that describes the case of a not contacting cutting device.

Alternatively or additionally, it can be that a second signal path is formed avoiding the fluid. In this way a reference value can be recorded that describes the case of a contacting cutting device.

Preferably the cutting device is deactivated in order to prevent tissue damage. The previously described advantages in connection with the relevant embodiment of the cutting device can thus be utilized as part of a method according to the invention, more particularly as described in the following.

In an advantageous embodiment it can be that a reference value for an impedance between the distal end of the shaft and a or the measuring electrode is determined when the distal end of the shaft contacts the wall of the hollow organ.

Alternatively or additionally, it can be that the determination of a reference value takes place when the distal end of the shaft is located freely within the hollow organ.

The impedance can be that between the proximity sensor and a or the measuring sensor. Here, it can be that the distal end of the shaft is surrounded by at least one of the electrically weakly conductive and non-conductive fluids.

As previously described in connection with the cutting device, this results in a resistance value moving from one reference value towards the other reference value which can be interpreted as an approach.

The invention will now be described in more detail by way of one example of embodiment but is not limited to the example of embodiment. Further examples of an embodiment result from combining the features of an individual feature or several features of the example of embodiment.

### BRIEF DESCRIPTION OF THE DRAWINGS

FIG. 1 shows a schematic view of a cutting device according to the invention during its intended use.

FIG. 2 shows a cross-sectional view through an endoscope including a cutting device according to the invention in accordance with FIG. 1.

FIG. 3 shows a schematic view of a further cutting device according to the invention during its intended use.

### DETAILED DESCRIPTION OF THE INVENTION

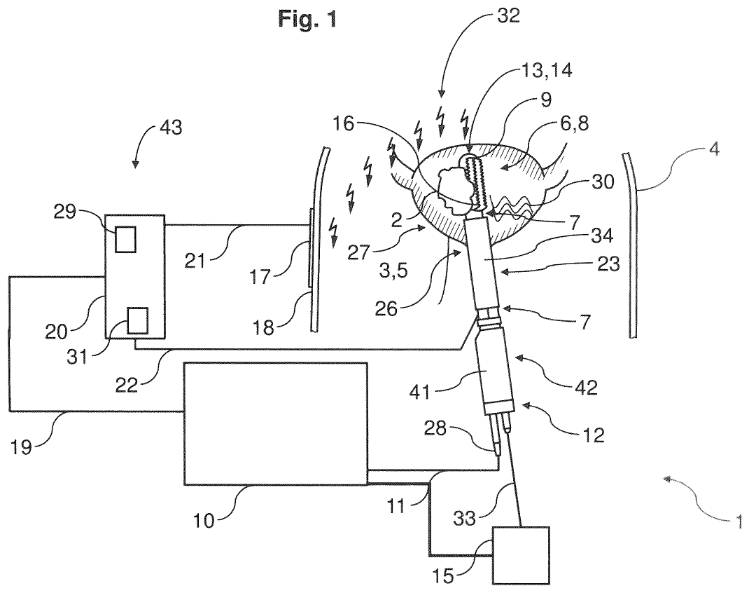

FIG. 1 shows the use of a cutting device 1 according to the invention as part of a method according to the invention for comminuting biological material 2 within a hollow organ 3 of a human patient 4. In this case the hollow organ 3 is the bladder 5. At the distal end 5 of a shaft 7, the cutting device 1 comprises a mechanically drivable cutting head 8. The cutting head 8 comprises a blade 9. To carry out a cutting movement, the blade 9 can be driven with a drive 42 positioned in a handle 41 of the cutting device 1.

Furthermore, a control unit 10 is connected to the proximal end 12 of the shaft 7 via an operating current circuit 11. The control unit 10 is for operating the drive 42, in particular for switching it off.

A probe 13 of a proximity sensor 43 is also formed on the distal end 6 of the shaft 7. In this case, the probe 13 is a conductive section 14 on the distal end 6 of the shaft 7. The conductive section 14 is a metallic part of the shaft 7 that forms the probe 13. The shaft 7 is made of metal, and the metallic part 13 that forms the probe 13 is an integral part of the exterior of the shaft 7. The metallic part of the shaft 7 forms the distal end 6 of the shaft 7. The metallic part of the shaft also provides mechanical stability of the shaft 7 and accommodates the blade 9 of the cutting head 8 in a housing.

The control unit 10 controls and switches off the cutting head 8 in the event of an approach, more particularly contact, detected by the proximity sensor.

In addition, a suction device 15 is fluidically connected via a suction line 33, extending at least partially through the interior of the shaft 7, to a suction opening 16 formed at the distal end 6 of the shaft 7.

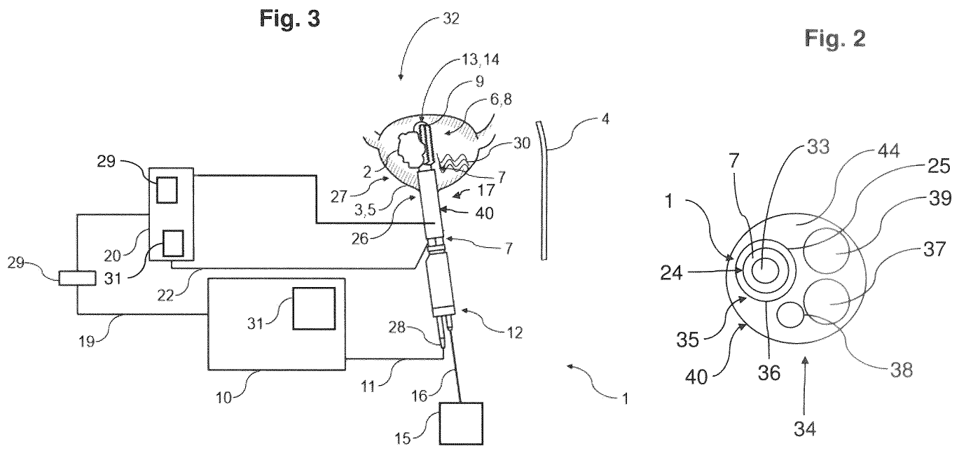

Also provided is a rinsing channel 39 or rising line, shown in more detail in FIG. 2, for emitting a fluid from the distal end 6 of the shaft 7. The rinsing line runs outside the shaft 7 in an endoscope shaft 44.

The control unit 10 also controls and switches off the suction device 15 in the event of an approach detected by the proximity sensor 13.

For this, the control unit 10 controls the cutting head 8 in reverse in the event of an approach detected by the proximity sensor 13. In doing so, the blade 9 of the cutting head 8 is moved backwards and in the direction of a neutral position.

The control unit 10 also controls the suction device 15 in reverse in the event of an approach detected by the proximity sensor 13. In doing so, the suction device 15 emits a fluid at its suction opening 16.

The control unit 10 is also connected to a measuring device 20 by way of a control connection 19.

The control unit 10 and measuring device 20 can be designed separately or provided as an integrated device.

The measuring device 20 is connected to a measuring electrode 17. The measuring electrode 17 is applied to a body part 18. In the present illustration, it is applied in the pelvic area. In this case, the connection between the measuring device 20 and measuring electrode 17 is implemented via a measuring current line 21 that is connected to the electrode 17. A further measuring current line 22 is connected to the shaft 7 of the cutting device 1. In this way a current flow 32 between the probe 13 and the measuring electrode 17 is possible.

In one section 23, the shaft 7 is also electrically insulated vis-a-vis the surroundings (cf. FIG. 2). For this, an electrical insulation 25 is formed on the outer side 24 of the shaft 7, for instance with shrink tubing or a polymer coating.

When the cutting device 1 is being used as intended, the section 23 is at least at the level of an access opening 26 of an intervention space 27. Here, the intervention space 27 is the interior of the hollow organ 3 in which the surgical intervention takes place.

In general, the cross-sectional view according to FIG. 2 shows an endoscope 34, for example a resectoscope, with an operating channel 35 that is formed in an endoscope shaft 44 and in which the cutting device 1, for example a morcellator, is arranged in a displaceable manner.

The endoscope 34 also has a lens 37, lighting 3 for the lens 37 and a rinsing channel 39.

In a further example, the insulation 25 is formed on the inner side 36 of the operating channel 35 of the endoscope 34.

In a further example of embodiment (not shown), the insulation 25 is formed on the outer side 40 of the endoscope 34.

Also formed on the proximal end 12 of the shaft 7 according to FIG. 1, is a connection 28 for diverting a current from the distal end 6 of the shaft 7. The shaft 7 is electrically connected to the control unit 10 via the operating current circuit 11. A generator 29 provides high-frequency alternating current which is capable of generating, for example, an alternating current with a frequency of between 30 and 35 kHz. Direct current generators or alternating current generators with other frequency ranges can also be used.

The cutting device 1 is configured to determine an electrical resistance, in this case the impedance, between the distal end 6 of the shaft 7, more specifically between the proximity sensor 13 and the measuring electrode 17, for detecting an approach.

The distal end 6 of the shaft 7 of the cutting device 1 is also surrounded by an electrically weakly conductive to non-conductive fluid 30. The fluid 30 is present in the form of a liquid which is shown in a stylized manner in the shape of waves. The fluid 30 is in contact with the biological tissue of the hollow organ 3. The cutting device 1 also comprises a memory unit 31, which in the present example of embodiment is the control unit 10 and which stores a reference value for an impedance between the distal end 6 of the shaft 7 and the measuring electrode 17. The reference value is then used for detecting the approach.

With the cutting device 1 according to the invention, two reference values can even be determined. These two reference values can be generated with the distal end 6 of the shaft 7 being advanced so far that the distal 6 of the shaft 7 contacts the tissue of the hollow organ 3 and a first reference value is determined, and a further reference value is then determined when the distal end 6 of the shaft 7 is located freely within the hollow organ 3 and thus surrounded by the electrically weakly conductive to non-conductive fluid 30.

FIG. 3 shows a further example of embodiment. Components and functional units that are similar or identical in function or design to the preceding example of embodiment are shown with the same reference numbers and are not described separately again. The details relating to FIGS. 1 and 2 thus apply to FIG. 3 accordingly.

The example of embodiment according to FIG. 3 differs from the preceding examples of embodiment in that an outer side 40 of the endoscope 34 is used as the measuring electrode 17.

In the context of using the cutting device 1 according to the invention, the biological material 2 is comminuted by way of the cutting head 8 and sucked in by the suction device 15 and thus transported away out of the hollow organ 3.

Thus a probe 13 is provided having a proximity sensor 43 on a cutting device 1 with a mechanically driveable cutting head 8 formed at a distal end 6 of a shaft 7 and a control unit 10 at the distal end 6 of the shaft 7. The cutting device 1 is used as part of method for comminuting biological material 2 within a hollow organ 3.

### LIST OF REFERENCE NUMBERS

1 Cutting device
2 Biological material
3 Hollow organ
4 Patient
5 Bladder
6 Distal end
7 Shaft
8 Cutting head
9 Blade
10 Control unit for drive and/or suction device
11 Operating current circuit
12 Proximal end
13 Probe of the proximity sensor
14 Section
15 Suction device
16 Suction opening
17 Measuring electrode
18 Body part
19 Control current connection
20 Measuring device
21 Measuring current line
22 Measuring current line
23 Section
24 Outer side
25 Insulation
26 Access opening
27 Intervention space
28 Connection
29 Generator
30 Fluid
31 Memory unit
32 Current flow
33 Suction line
34 Endoscope
35 Operating channel
36 Inner side
37 Lens
38 Lighting
39 Rinsing channel
40 Outer side
41 Handle
42 Drive
43 Proximity sensor
44 Endoscope shaft

1-15. (canceled)

16. A cutting device, comprising:
a driveable cutting head formed at a distal end of a shaft including a metallic part, a probe including a proximity sensor located at the metallic part of the distal end of the shaft which senses approach of the cutting head to an organ, and a control for operating the driveable cutting head to cause the cutting head to approach the organ.

17. The cutting device according to claim 16, wherein the metallic part of the distal end of the shaft mechanically stabilizes the shaft, accommodates a blade of the cutting head in a housing, and the control controls switching on and off of the cutting blade based on the proximity sensor detecting the cutting head is approaching contacting the organ.

18. The cutting device according to claim 16, comprising a suction device in fluidic connection with a suction opening located at the distal end of the shaft, the fluidic connection extending through an interior of the shaft and the control controls at least one controlling operation of the cutting blade and switching on and off the suction device based on the proximity sensor detecting that the cutting head is approaching contacting the organ.

19. The cutting device according to claim 17, comprising a suction device in fluidic connection with a suction opening located at the distal end of the shaft, the fluidic connection extending through an interior of the shaft and the control controls at least one controlling operation of the cutting blade and switching on and off the suction device based on the proximity sensor detecting that the cutting head is approaching contacting the organ.

20. The cutting device according to claim 16, wherein the control reverses the cutting head when the proximity sensor senses the cutting head is approaching the organ and the cutting head is controlled by the control to at least one of moving the cutting head backwards away from the organ and moving in a direction toward a neutral position relative to the organ.

21. The cutting device according to claim 19, wherein the control reverses the cutting head when the proximity sensor senses the cutting head is approaching the organ and the cutting head is controlled by the control to at least one of move the cutting head backwards away from the organ and in a direction toward a neutral position relative to the organ, and the control controls reversing of a direction of suction when the cutting head is detected by the proximity sensor as approaching contacting the organ while the suction device is emitting fluid at a suction opening of the section device or sucking fluid within the opening.

22. The cutting device according to claim 16, comprising a measuring device which controls the control is connected to a measuring electrode applied to a body part.

23. The cutting device according to claim 16, comprising a section of the shaft which is electrically insulated from surroundings, and wherein on an inner or an outer side of an endoscopic tube electrical insulation surrounds the shaft at an access opening to the shaft at an intervention space to the endoscopic tube.

24. The cutting device according to claim 17, comprising an endoscopic tube, a measuring device which is connected to a measuring electrode applied to a body part and a section of the shaft which is electrically insulated from surroundings, and wherein on an inner or an outer side of the endoscopic tube electrical insulation surrounds the shaft at an access opening to the shaft at an intervention space to the endoscopic tube.

25. The cutting device according to claim 16, comprising an electrical connection at a proximal end of the shaft for diverting electrical current flowing from the distal end of the shaft, the shaft is electrically connected to the control and the proximity sensor, and a generator provides alternating current, in a frequency between 30 Hz and 35 kHz, which is connected to the shaft.

26. The cutting device according to claim 16, wherein an electrical impedance is located between the distal end of the shaft and the proximity sensor and the electrical impedance is used for detecting approach of the proximity sensor to the organ and the proximity sensor uses a measuring electrode for measuring an alternating current flowing from the shaft to the proximity sensor.

27. The cutting device according claim 16, wherein the distal end of the shaft is surrounded by at least one of an electrically conductive fluid and non-electrically conductive fluid.

28. The cutting device according to claim 16, comprising a memory which stores at least one reference impedance that is disposed between the distal end of the shaft comprising the proximity sensor and a measuring electrode, and the at least one reference impedance is used for detecting approach of the cutting head to the organ.

29. A method for comminuting biological material within a hollow organ comprising using the cutting device of claim 16.

30. The method according to claim 29, wherein biological material is comminuted by the cutting head and is sucked in by the suction device from the organ and is transported away from the hollow organ through the suction opening.

31. The method according to claim 29, wherein measuring electrode is applied from outside an abdominal area of a patient.

32. The method according to claim 29, wherein the hollow organ is at least partially filled with a fluid and then is completely filled with at least one of an electrically conductive and non-conductive fluid. and the hollow organ is completely filled with at least one of the fluids.

33. The method according to claim 26, wherein at least two reference values are determined of an impedance which is disposed between the distal end of the shaft at the proximity sensor which is used by the measuring electrode.

34. The method according to claim 26, wherein a reference value of an impedance disposed between the probe and the measuring electrode are determined when the distal end of the shaft contacts at least one of a wall of the hollow organ and a distal end of the shaft.
